(19) United States
(12) Patent Application Publication
(10) Pub. No.: US 2026/0207737 A1
(43) Pub. Date: Jul. 23, 2026

(54) OIL ADJUVANT COMPOSITIONS AND VACCINE EMULSIONS COMPRISING SUCH OIL ADJUVANT COMPOSITIONS

(71) Applicant: CEVA SANTE ANIMALE, LIBOURNE (FR)

(72) Inventors: Jérôme Gaucheron, LIBOURNE (FR); Laura Svajda, LIBOURNE (FR)

(21) Appl. No.: 19/140,857

(22) PCT Filed: Dec. 21, 2023
(86) PCT No.: PCT/EP2023/087232

§ 371 Date: Jun. 18, 2025

(30) Foreign Application Priority Data
Dec. 22, 2022 (EP) 22306991.5

(51) Int. Cl. A61K 39/39; A61K 39/00; A61K 39/02; A61K 39/09; A61K 39/12; A61P 31/04; A61P 31/20

(52) U.S. Cl. CPC A61K 39/39; A61K 39/0241; A61K 39/092; A61K 39/12; A61P 31/04; A61P 31/20; A61K 2039/552; A61K 2039/55566; C12N 2750/10034

(57) ABSTRACT

The invention relates to oil adjuvant compositions comprising at least one oil, at least one nonionic lipophilic surfactant, which is a sorbitan ester or a mannitan ester, and at least one nonionic hydrophilic surfactant, which is an ethoxylated fatty alcohol. The invention also relates to adjuvant oil-in-water emulsions and vaccines oil-in-water emulsions obtained from the oil adjuvant compositions of the invention, and to the use of such vaccine oil-in-water emulsions for the treatment of porcine circovirus type 2 disease and/or porcine enzootic pneumonia.

### TECHNICAL FIELD

The invention relates to oil adjuvant compositions comprising specific surfactants, that are especially suitable for formulating vaccine oil-in-water emulsions.

### TECHNICAL BACKGROUND

The use of adjuvants in pharmaceutical compositions, such as vaccine compositions, is well known in the art. Generally, emulsions used in vaccine formulation comprise a mixture of a fatty phase, an aqueous phase and at least one surfactant.

Two main criteria for selecting suitable adjuvants when formulating a pharmaceutical composition, especially a vaccine emulsion, are stability and safety, especially in presence of bacterial and/or viral suspensions. For instance, stability of the adjuvant may be impacted because of interaction of the component of the emulsion with the lipase or esterase enzymatic activities present in the antigen suspension of the vaccine. In particular, this issue may be observed when the vaccine is based on inactivated or attenuated pathogens or on a combination of pathogens. In addition, safety issues may arise due to the toxicity of some surfactants present in adjuvants.

WO 2005/009462 discloses oil-in-water emulsions comprising an aqueous phase comprising an immunogen, and an oily phase with at least three surfactants: a non-ionic lipophilic surfactant, which is a fatty ester of sorbitan; a non-ionic hydrophilic surfactant, which is an ethoxylated sorbitan fatty acid monoester, and a non-ionic hydrophilic surfactant, which is sorbitan fatty acid triester. Said adjuvant compositions are disclosed to be suitable for forming vaccine compositions with increased safety and stability. However, the exclusive use of sorbitan ester surfactants may affect the stability of the oil-in-water emulsions, especially when the adjuvant compositions are used with an antigen produced from a cell line comprising enzymes such as lipases or esterases. For instance, the cell line used to produce the “Mycoplasma hyo” antigen may contain enzymes such as esterases which may destabilize the emulsion made with an adjuvant composition comprising only ester surfactants, in particular ethoxylated ester surfactants.

WO 2006/113373 discloses oil-in-water adjuvant formulations comprising two surfactants, one non-ionic lipophilic ethoxylated fatty alcohol and one non-ionic hydrophilic ethoxylated fatty alcohol. This formulation uses exclusively ethoxylated fatty alcohol surfactants that may lead to a decrease in the stability of the formulation and pose more safety concerns due to the toxicity and ecotoxicity of this family of emulsifiers. Actually, ethoxylated fatty alcohol surfactants comprise ether functions, which cannot be reversed and cannot be degraded by enzymes such as esterases. Said surfactants are thus poorly bio adaptable by enzymes and/or pH changes, which contributes to their toxicity (see for instance Oda et al. Research in Veterinary Science 81 (2006), p. 51-57). The exclusive use of such surfactants in a composition thus increases safety concerns in terms of toxicity of said composition. WO2006/113373 discloses that the claimed formulations present an increased safety and stability. No stability results are however presented in this patent application.

Thus, there remains a need to provide novel adjuvant compositions with increased stability and safety, even when the antigen may comprise additional components such as enzymes.

### SUMMARY OF THE INVENTION

In this respect, the Inventors have unexpectedly demonstrated that the use of a specific combination of surfactants comprising at least one nonionic lipophilic surfactant, which is a sorbitan ester or a mannitan ester, and at least one nonionic hydrophilic surfactant, which is an ethoxylated fatty alcohol, affords oil adjuvant compositions with increased stability and safety, even when used in a vaccine emulsion with an antigen that comprises additional components such as enzymes.

Thus, the present invention first relates to an oil adjuvant composition comprising:
at least one oil,
at least one nonionic lipophilic surfactant, which is a sorbitan ester or a mannitan ester, and
at least one nonionic hydrophilic surfactant, which is an ethoxylated fatty alcohol.

In an embodiment, the oil comprises at least one mineral oil, in particular at least one light paraffin oil, or a combination of at least two mineral oils.

In an embodiment, the mineral oil is selected from the group consisting of paraffin oil such as isoparaffinic oil and/or naphthenic oil, pristane, and any mixture thereof.

In an embodiment, each sorbitan ester or mannitan ester is a sorbitan ester, wherein the sorbitan ester is preferably selected from the group consisting of sorbitan monolaurate, sorbitan monopalmitate, sorbitan monostearate, sorbitan tristearate, sorbitan monooleate, sorbitan trioleate, sorbitan monoisostearate, and sorbitan isostearate, more preferably is sorbitan monooleate.

In another embodiment, each sorbitan ester or mannitan ester is a mannitan ester, preferably selected from the group consisting of mannitan oleate, mannitan monooleate, mannitan dioleate, mannitan trioleate and mannitan tetraoleate.

In an embodiment, the ethoxylated fatty alcohol is selected from the group consisting of polyoxyethylene (23) lauryl ether, polyoxyethylene (20) cetyl ether, polyoxyethylene (10) oleyl ether, polyoxyethylene (20) oleyl ether, polyoxyethylene (21) stearyl ether, polyoxyethylene lauryl ether, eicosaethylene glycol octadecyl ether, triethylene glycol oleyl ether, and any mixture thereof, preferably polyoxyethylene oleyl ether, in particular polyoxyethylene (10) oleyl ether.

In an embodiment, the oil adjuvant composition comprises:
from 76% to 92% of oil,
from 1% to 7% of at least one nonionic lipophilic surfactant, which is a sorbitan ester or a mannitan ester, and
from 7% to 17% of at least one nonionic hydrophilic surfactant, which is an ethoxylated fatty alcohol, wherein the percentages are expressed in weight relative to the total volume of the oil adjuvant composition.

In an embodiment, the oil adjuvant composition comprises:
about 82% of oil, preferably of mineral oil,
about 4% of at least one nonionic lipophilic surfactant, which is a sorbitan ester or a mannitan ester, preferably of sorbitan monooleate, and
about 14% of at least one nonionic hydrophilic surfactant, which is an ethoxylated fatty alcohol, preferably of polyoxyethylene oleyl ether, wherein the percentages are expressed in weight relative to the total volume of the oil adjuvant composition.

The present invention also relates to an adjuvant oil-in-water emulsion, comprising from 40% to 70% of an oil adjuvant composition according to the invention and from 30% to 60% of an aqueous solvent, the oil adjuvant composition being dispersed in the aqueous solvent, wherein the percentages are expressed in volume relative to the total volume of the adjuvant oil-in-water emulsion.

The present invention further relates to a vaccine oil-in-water emulsion, comprising an adjuvant oil-in-water emulsion according to the invention, at least one antigen, and a pharmaceutically acceptable carrier, preferably an aqueous pharmaceutically acceptable carrier.

In an embodiment, the vaccine oil-in-water emulsion comprises from 10 to 80% of adjuvant oil-in-water emulsion, and from 20% to 90% of pharmaceutically acceptable carrier, wherein the percentages are expressed in volume relative to the total volume of the vaccine oil-in-water emulsion.

In an embodiment, the at least one antigen is selected from the group consisting of an inactivated porcine circovirus type 2 (PCV2) antigen, a Mycoplasma hyopneumoniae antigen, a Streptococcus suis antigen and a combination thereof.

The present invention further relates to a vaccine oil-in-water emulsion according to the invention for use as a medicament, preferably for use as a vaccine.

A last object of the present invention is a vaccine oil-in-water emulsion according to the invention for use in the treatment of porcine circovirus type 2 disease, a Streptococcus suis infection such as porcine meningitis, porcine septicemia, porcine arthritis, porcine endocarditis, or porcine sudden death and/or porcine enzootic pneumonia.

In an embodiment, the vaccine oil-in-water emulsion is administered, preferably injected, to an animal being a pig or a piglet.

### BRIEF DESCRIPTION OF FIGURES

FIG. 1 presents photographs of the visual appearance of the compositions studied in example 2 after 1 day (a), after 1 week (b) and after 1 month (c).

FIGS. 2 to 5 present graphs of the size distribution of particles over time in the compositions studied in example 2. FIG. 2: Composition A; FIG. 3: Comparative composition B; FIG. 4: Composition LR3 and FIG. 5: Composition LR4.

FIG. 6 is a graph comparing the temperature elevation in the four groups of the safety study of example 4.

FIG. 7 presents histograms of the viremia of each group at day 14 (A), 21 (B) and 28 (C) after the challenge for the study of example 5.

FIG. 8 presents histograms of the log of nasal swab at day 7 (A), 14 (B), 21 (C) and 28 (D) for the study of example 5.

FIG. 9 presents histograms of the nasal swab at day 7 (A), 14 (B), 21 (C) and 28 (D) for the study of example 5.

FIG. 10 presents histograms of the log quantification of the virus at day 28 for respectively inguinal lymph nodes (A), mesenteric lymph nodes (B), lung (C), mediastinal lymph nodes (D) and tonsil lymph nodes (E) for the study of example 5.

FIG. 11 is a graph presenting the percentage of local reaction per day for the study of example 6.

FIG. 12 is a graph presenting antibody development for each group before the first vaccination (B0), between the first and the second vaccinations (B1) and 2 weeks after the second vaccination (B2) in the study of example 6.

### DETAILED DESCRIPTION OF THE INVENTION

### Definitions

“About” is used herein to mean approximately, roughly, around, or in the region of. When the term “about” is used in conjunction with a numerical range or value, it modifies that range or value by extending the boundaries up to 10%, preferably 5%, more preferably 1%, above and below the numerical range or value.

An “adjuvant composition” is a composition that is suitable for forming a pharmaceutical composition, such as a vaccine composition, when combined with at least an active agent, such as an antigen. An adjuvant composition increases the immune response to a vaccine antigen when compared to the response induced by the vaccine antigen alone. In some other non-preferred embodiments, the adjuvant composition does at least not reduce the immune response to a vaccine antigen when compared to the response induced by the vaccine antigen alone.

The term “animal” encompasses all vertebrate animals including humans. In particular, the term “vertebrate animals” includes humans, canines (such as dogs), felines (such as cats), equines (such as horses), bovines (such as cattle), porcines (such as pigs, swines or sows), ovines (such as sheeps), caprines (such as goats) and avians (such as chickens).

An “antigen” refers to a substance that induces a specific immune response in a host animal. An antigen may be an epitope, an immunogenic fragment or an entire organism (bacteria, virus) that has been inactivated, attenuated or genetically modified.

An “emulsion” refers to a colloidal system made of two non-miscible elements, for example oil and water, one element (the dispersed phase) being present in the form of droplets dispersed in the other element, constituting the continuous phase. An “oil-in-water emulsion” refers to an emulsion, wherein the dispersed phase is a fatty phase, such as oil, and the continuous phase is an aqueous phase, such as water.

The term “ethoxylated”, when used to qualify a compound comprising a labile hydrogen atom, such as a hydrogen atom of a hydroxy group, refers to a compound wherein said hydrogen labile group has been substituted with a —(C₂H₄O)ₙ—H moiety, n being 1 or more. n typically ranges from 1 to 20. The term ethoxylated may be used for instance to qualify a fatty alcohol (ethoxylated fatty alcohol) or a sorbitan or mannitan ester (ethoxylated sorbitan ester or ethoxylated mannitan ester).

The term “fatty”, when used to qualify a compound comprising a hydrocarbon chain, refers to a compound comprising an aliphatic hydrocarbon chain comprising from 4 to 36 carbon atoms, preferably from 4 to 28 carbon atoms, more preferably from 8 to 20 carbon atoms, in particular from 12 to 18 carbon atoms. The aliphatic hydrocarbon chain may be saturated or unsaturated. The term fatty may be used for instance to qualify an alcohol (fatty alcohol), an ester (fatty ester) and/or an acid (fatty acid).

A “mannitan ester” is a derivative obtained by esterification of at least one of the alcohol and/or phenol functions of mannitan.

A “mineral oil” is any of various colorless, odorless, light mixtures of higher alkanes from a mineral source, particularly a distillate of petroleum.

An “oil” is a fatty substance, which is fluid, preferably liquid, at room temperature (25-35° C.), and insoluble in water. It may be synthetic or of vegetable, animal or mineral origin.

The term “pharmaceutically acceptable carrier” refers to a fluid vehicle for containing a pharmaceutical active agent, such as an antigen, and that can be administered to an animal, preferably injected to an animal, without significant adverse effects. The pharmaceutically acceptable carrier does not interact with the pharmaceutical active agent, and does preferably not affect its pharmaceutical efficiency. Suitable pharmaceutically acceptable carriers known in the art include sterile water, saline, glucose, dextrose, and buffered solutions. Carriers may include auxiliary agents such as diluents, stabilizers, sugars, amino acids, preservatives, wetting agents, emulsifying agents, pH buffering agents, viscosity enhancing additives, colors or any mixture thereof.

The terms “swine”, “pig” and “piglet” refer to an animal of porcine origin. A “sorbitan ester” is a derivative obtained by esterification of at least one of the alcohol and/or phenol functions of sorbitan.

“Treating” or “treatment” refers to both therapeutic treatment and prophylactic or preventive measures, wherein the object is to prevent or slow down (lessen) the targeted pathologic condition or disorder. Those in need of treatment include those already with the disorder as well as those prone to have the disorder or those in whom the disorder is to be prevented. A subject or animal is successfully “treated” for an infection if, after receiving a therapeutic amount of a composition according to the present invention, the subject shows observable and/or measurable reduction in or absence of one or more of the following: reduction in the number of pathogenic cells; reduction in the percent of total cells that are pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.

### Oil Adjuvant Composition

A first object of the invention is an oil adjuvant composition comprising
at least one oil,
at least one nonionic lipophilic surfactant, which is a sorbitan ester or a mannitan ester, and
at least one nonionic hydrophilic surfactant, which is an ethoxylated fatty alcohol.

The Inventors demonstrated that the oil adjuvant compositions and the vaccine oil-in-water emulsions according to the invention are highly stable, even when the antigen may comprise additional elements such as enzymes, and present a high safety. The stability of the oil adjuvant compositions and vaccine oil-in-water emulsions has even be proven to be higher than that of formulations disclosed in prior art to be highly stable.

Oils are known to be suitable adjuvants for active agents as they are immunostimulant. The oil compositions according to the invention are suitable as adjuvant, especially for veterinary compositions. Furthermore, oil-in-water emulsions are known to be suitable as adjuvants, especially for veterinary compositions, as disclosed in publications such as Burakova et al. Viral Immunology 31, 1, 2018, p. 11-22.

The oil adjuvant composition according to the invention comprises at least one non-ionic hydrophilic surfactant, which is an ethoxylated fatty alcohol, and at least one non-ionic lipophilic surfactant, which is a sorbitan ester or a mannitan ester.

In some embodiments, the at least one non-ionic hydrophilic surfactant and the at least one non-ionic lipophilic surfactant are the only surfactants comprised in the oil adjuvant composition according to the invention. In some embodiments, the oil adjuvant composition according to the invention comprises as only surfactants one ethoxylated fatty alcohol and one sorbitan ester or mannitan ester.

A hydrophilic ethoxylated fatty alcohol may comprise from 1 to 50, preferably from 5 to 30, ethylene oxide moieties. The fatty alcohol may be a C4 to C36 fatty alcohol, preferably a C9 to C22 fatty alcohol, and is advantageously selected from the group consisting of oleyl, cetyl, stearyl, isostearyl, lauryl and octadecyl alcohols, and combinations thereof, advantageously it is an oleyl alcohol. More advantageously, the ethoxylated fatty alcohol is an oleyl alcohol with 3 to 20 ethylene oxide moieties.

The ethoxylated fatty alcohols may be for instance selected from the group consisting of: polyoxyethylene (23) lauryl ether, such as Brij 76®, polyoxyethylene (20) cetyl ether, such as Brij 56® or Brij 58®, polyoxyethylene (10) oleyl ether, such as Brij 96/97® or Brij 010®, polyoxyethylene (20) oleyl ether, such as Brij 98® or Brij 020®, polyoxyethylene (21) stearyl ether, such as Brij 721®, polyoxyethylene lauryl ether, such as Brij 35®, eicosaethylene glycol octadecyl ether, such as Brij 78®, triethylene glycol oleyl ether, such as Volpo N5®, and any mixture thereof.

In some embodiments, the ethoxylated fatty alcohols are used as mixtures.

In some embodiments, the ethoxylated fatty alcohols are selected from those with the following brand names: Ceteareth-6® (68439-49-6), Ceteareth-12® (68439-49-6), Ceteareth-20® (68439-49-6), Ceteareth-25® (68439-49-6), Ceteareth-23® (68439-49-6), Laureth-9®, Laureth-21®, Laureth-25®, Ceteth-6®, Ceteth-7®, Ceteth-15®, Ceteth-23®, Ceteth-25®, C12-14 Pareth-5®, C12-14 Pareth-7®, C12-14 Pareth-9®, C12-14 Pareth-12®, C12-15 Pareth-10®, Oleth-7®, Oleth-15®, Oleth-50®, Beheneth-20® (Nikko Chemicals), and any combination thereof.

The ethoxylated fatty alcohols preferably present a hydrophilic-lipophilic balance (HLB) comprised between 10 and 18, preferably between 12 and 18.

The surfactants of the invention may comprise fatty alcohols from animal or vegetable origin. The amount of the at least one ethoxylated fatty alcohol in the oil adjuvant compositions according to the invention may vary in a wide range and may be determined by one skilled in the art depending, among others, on the chemical nature of the at least one ethoxylated fatty alcohol, the nature of the other components (oil and/or sorbitan or mannitan ester), and/or the nature of the pharmaceutical active agent, especially the antigen, that will be present in the vaccine oil-in-water emulsion comprising the oil adjuvant composition.

The ethoxylated fatty alcohol(s) may be present in the oil adjuvant composition in an amount ranging from about 5 to about 50% of the oil adjuvant composition, preferably from about 7 to about 17% of the oil adjuvant composition, the percentage being expressed in weight relative to the total volume of the oil adjuvant composition.

The non-ionic lipophilic surfactant is a non-ethoxylated sorbitan ester or a non-ethoxylated mannitan ester. Ethoxylated sorbitan and mannitan esters comprising more than six ethylene oxide moieties are hydrophilic.

The non-ionic lipophilic surfactant is an ester of sorbitan or of mannitan, preferably a fatty ester of sorbitan or of mannitan. In some embodiments, each non-ionic lipophilic surfactant is a sorbitan ester, preferably a fatty ester of sorbitan, more preferably a monoester of fatty acid of sorbitan or a triester of fatty acid of sorbitan. In other embodiments, each non-ionic lipophilic surfactant is a mannitan ester, preferably a fatty ester of mannitan.

Examples of fatty esters of sorbitan include sorbitan monolaurate, such as Span 20®, sorbitan monopalmitate, such as Span 40®, sorbitan monostearate, such as Span 60®, sorbitan tristearate, such as Span 65®, sorbitan monooleate, such as Span 80®, sorbitan trioleate, such as Span 85®, sorbitan monoisostearate, such as Arlacel 987® and sorbitan isostearate, such as Crill 6®.

Examples of fatty esters of mannitan include mannitan oleate, such as Montanide 80®, mannitan monooleate, such as Arlacel A®, mannitan dioleate, mannitan trioleate and mannitan tetraoleate.

The fatty acids used for forming the fatty alcohols and/or the fatty esters implemented in the present invention are preferably selected from the group consisting of oleic acid, palmitic acid, stearic acid, isostearic acid, lauric acid and combinations thereof.

The amount of the at least one sorbitan ester or mannitan ester in the oil adjuvant compositions according to the invention may vary in a wide range and may be determined by one skilled in the art depending, among others, on the chemical nature of the at least one sorbitan or mannitan ester, the nature of the other components (oil and/or ethoxylated fatty alcohol), and/or the nature of the pharmaceutical active agent, especially the antigen, that will be present in the vaccine oil-in-water emulsion comprising the oil adjuvant composition.

The at least one sorbitan ester or mannitan ester may be present in the oil adjuvant composition in an amount ranging from about 0.1 to about 25% of the oil adjuvant composition, preferably from about 1 to about 7% of the oil adjuvant composition, the percentage being expressed in weight relative to the total volume of the oil adjuvant composition.

The oil may be a synthetic oil, or an oil of vegetable, animal or mineral origin.

The oil may be a mineral oil including, but not limited to, paraffin oil such as isoparaffinic oil and/or naphthenic oil, pristane, and any mixture thereof. One advantageous mineral oil useful in the present invention may include an oil comprising a linear or ramified carbon chain having a number of carbon atoms greater than 15, preferably from 15 to 32, and free of aromatic compounds. The oil may, for example, be one of those marketed under the name MARCOL 52® or MARCOL 82® (produced by Esso, France), DRAKEOL 6VR® (produced by Penreco, USA), or EOLANE 150®, EOLANE 170® or EOLANE 130® (produced by TotalEnergies). The mineral oil may be selected from the group consisting of an oil of CAS number 8042-47-5 (white mineral oil), an oil of CAS number 8012-95-1 (light liquid paraffin), an oil of CAS number 8020-83-5 (mineral oil), and any mixture thereof.

The oil may be a synthetic oil including, but not limited to, squalane, polyisobutene oil, hydrogenated polyisobutene oil, polydecene oil, polyisoprene oil, polyisopropene oil and the like, and any mixture thereof.

In an embodiment, the oil comprised in the oil adjuvant composition according to the invention comprises at least one mineral oil, in particular at least one light paraffin oil, or a combination of at least two mineral oils.

In an embodiment, the oil comprised in the oil adjuvant composition according to the invention consists of at least one mineral oil, in particular at least one light paraffin oil, or a combination of at least two mineral oils.

The oil may also be a mixture of oils comprising at least two oils selected among the oils described herein, in any proportion.

The amount of oil in the oil adjuvant compositions according to the invention may vary in a wide range and may be determined by one skilled in the art depending, among others, on the chemical nature of the oil, the nature of the other components (sorbitan or mannitan ester and/or ethoxylated fatty alcohol), and/or the nature of the pharmaceutical active agent, especially the antigen, that will be present in the vaccine oil-in-water emulsion comprising the oil adjuvant composition.

Oil may be present in the oil adjuvant composition in an amount ranging from about 50 to about 95% of the oil adjuvant composition, preferably from about 76 to about 92% of the oil adjuvant composition, the percentage being expressed in weight relative to the total volume of the oil adjuvant composition.

In some embodiments, the oil adjuvant composition according to the invention comprises a mixture of liquid saturated hydrocarbons, preferably MARCOL 52® oil, sorbitan monooleate and polyoxyethylene (10) oleyl ether.

In some embodiments, the oil adjuvant composition according to the invention comprises
from 76% to 92%, preferably about 82%, of oil, preferably of mineral oil,
from 1% to 7%, preferably about 4%, of at least one nonionic lipophilic surfactant, which is a sorbitan ester or a mannitan ester, preferably sorbitan monooleate, and
from 7% to 17%, preferably about 14%, of at least one nonionic hydrophilic surfactant, which is an ethoxylated fatty alcohol, preferably polyoxyethylene oleyl ether,
wherein the percentages are expressed in weight relative to the total volume of the oil adjuvant composition.

The oil adjuvant composition according to the invention may be prepared by any suitable process known in the art, for instance by using a mixer or an emulsifier.

As an example, the following process (preparation process A) may be used for preparing an oil adjuvant composition according to the invention, preferably an oil adjuvant composition comprising 76 to 92% of light liquid paraffin (CAS 8012-95-1), 1 to 7% of sorbitan monooleate (CAS 338-43-8), 7 to 17% polyoxyethylene 10 oleyl ether (CAS 9004-98-2), and sufficient water to obtain a clear composition.

Preparation process A comprises the following steps:
i. measuring the suitable amounts of oil, preferably light liquid paraffin, of sorbitan or mannitan ester, preferably sorbitan monooleate, and of ethoxylated fatty alcohol, preferably polyoxyethylene 10 oleyl ether;
ii. mixing thoroughly with a suitable mixer;
iii. adding water until the mixture is clear;
iv. autoclaving the mixture of step iii in a closed vessel at a temperature comprised between 110° C. and 130° C., for instance at 121° C., for a duration comprised between 30 minutes and 1 hour, for instance for 40 minutes; or
iv. filtering the mixture of step iii through a 0.22 μm filter.

### Adjuvant Oil-In-Water Emulsion

A second object of the invention is an adjuvant oil-in-water emulsion, which oily phase comprises, preferably consists of, an oil adjuvant composition according to the invention.

In an embodiment, the adjuvant oil-in-water emulsion according to the invention comprises from 40% to 70%, preferably from 40% to 60%, of an oil adjuvant composition according to the invention, and from 30% to 60%, preferably from 40% to 60%, of an aqueous solvent. The oil adjuvant composition is dispersed in the aqueous solvent. The percentages are expressed in volume relative to the total volume of the adjuvant oil-in-water emulsion.

The aqueous solvent of the adjuvant oil-in-water emulsion may be for instance selected from the group consisting of water, a physiological solution, such as a 0.9% NaCl aqueous solution, and an aqueous buffer, such as a PBS (phosphate buffered saline) buffer. In some embodiments, the aqueous solvent is water.

The adjuvant oil-in-water emulsion according to the invention may be prepared by any suitable process known in the art.

As an example, the following process (preparation process B) may be used for preparing an adjuvant oil-in-water emulsion according to the invention.

Preparation process B comprises the following steps:
i′) preparing an oil adjuvant composition according to the invention, preferably by preparation process A detailed above;
ii′) measuring one part of oil adjuvant composition (50% vol) and one part of aqueous solvent (50% vol), preferably of sterile PBS, under sterile conditions;
iii′) heating both phases, for instance at 70° C.;
iv′) while stirring, adding the aqueous phase to the oil phase;
v′) while continuing stirring, gradually cooling down the emulsion; and
vi′) storing the obtained emulsion between 2° C. and 8° C. until use.

Unless differently stated, in the present application, the percentages are expressed in volume relative to the total volume of the adjuvant oil-in-water emulsion.

The present invention also relates to a diluted oil-in-water emulsion, comprising an adjuvant oil-in-water emulsion according to the invention and a pharmaceutically acceptable carrier, preferably an aqueous pharmaceutically acceptable carrier. Said diluted oil-in-water emulsion may be used to be contacted with an active agent, such as an antigen, or with a composition, preferably an oil-in water emulsion, comprising such active agent.

### Vaccine Oil-In-Water Emulsion

A third object of the invention is an oil-in-water emulsion, preferably a vaccine oil-in-water emulsion, which comprises an adjuvant oil-in-water emulsion according to the invention, a further pharmaceutically acceptable carrier, preferably aqueous pharmaceutically acceptable carrier, and at least one active agent, such as an antigen.

Even if the present oil-in-water emulsions are particularly suitable for being used with antigens, for forming vaccine emulsions, they may also present advantages when used with different active agents.

The antigen may comprise a whole organism, killed, attenuated or alive; a subunit or portion of an organism; a recombinant vector containing an insert with immunogenic properties; a piece or fragment of DNA capable of inducing an immune response upon presentation to a host animal; a protein, a polypeptide; a peptide; an epitope; a hapten, or any combination thereof. Alternatively, the antigen may comprise a toxin or an antitoxin. The antigen may be inactivated.

In some embodiments, the antigen is an antigen from a pathogen associated with a swine disease, preferably selected from the antigens of virus and bacteria associated with a swine disease.

The viral antigen from a virus associated with swine disease may be selected from the group consisting of a classical swine fever virus antigen, a swine influenza virus antigen; a porcine reproductive and respiratory syndrome (PRRS) virus antigen, a porcine respiratory corona virus such as porcine epidemic diarrhea virus (PEDV) antigen, a porcine parvovirus antigen, a porcine rotavirus antigen and a porcine circovirus, such as porcine circovirus type 2 (PCV2), antigen. Preferably, the antigen from a virus associated with swine disease is a porcine circovirus type 2 (PCV2) antigen, such as ORF2 PCV2 antigen.

The bacterial antigen from a bacteria associated with a swine disease may be selected from the group consisting of: an Actinobacillus pleuropneumoniae antigen, a Mycobacterium spp. antigen, preferably Mycoplasma hyopneumoniae (M hyo) antigen, a Streptococcus suis antigen, preferably an IgM protease antigen, more preferably IdeSsuis antigen, a Lawsonia intracellularis antigen, an Escherichia coli antigen, an Eperythrozoonosis suis antigen, an Erysipelothrix rhsiopathiae antigen, a Bordetella bronchiseptica antigen, a Pasteurella multocida antigen, a Salmonella spp. antigen, a Haemophilus parasuis antigen, a Clostridium spp., such as Clostridium perfringens antigen, and a Leptospira spp. antigen. Preferably, the antigen from a bacteria associated with swine disease is a IdeSsuis antigen, a Lawsonia intracellularis antigen or a Mycoplasma hyopneumoniae (M hyo) antigen.

The bacterial antigen from a bacteria associated with a swine disease may be selected from the group consisting of: an Actinobacillus pleuropneumoniae antigen, a Mycobacterium spp. antigen, preferably Mycoplasma hyopneumoniae (M hyo) antigen, an Escherichia coli antigen, an Eperythrozoonosis suis antigen, an Erysipelothrix rhsiopathiae antigen, a Bordetella bronchiseptica antigen, a Pasteurella multocida antigen, a Salmonella spp. antigen, a Haemophilus parasuis antigen, a Clostridium spp., such as Clostridium perfringens antigen, and a Leptospira spp. antigen. Preferably, the antigen from a bacteria associated with swine disease is a Mycoplasma hyopneumoniae (M hyo) antigen.

In an embodiment, the at least one antigen comprised in the vaccine oil-in-water emulsion according to the invention is selected from the group consisting of a porcine circovirus type 2 (PCV2) antigen, a Streptococcus suis antigen, a Mycoplasma hyopneumoniae antigen and a combination thereof. More preferably, the at least one antigen comprised in the vaccine oil-in-water emulsion according to the invention is selected from the group consisting of a porcine circovirus type 2 (PCV2) antigen, a Mycoplasma hyopneumoniae antigen and a combination thereof.

In an embodiment, the at least one antigen comprised in the vaccine oil-in-water emulsion according to the invention is a combination of a porcine circovirus type 2 (PCV2) antigen, such as ORF2 PCV2 antigen, of a Mycoplasma hyopneumoniae antigen and of a Lawsonia intracellularis antigen.

The vaccine oil-in-water emulsion according to the invention may comprise from 10 to 80% of adjuvant oil-in-water emulsion, and from 20% to 90% of pharmaceutically acceptable carrier, wherein the percentages are expressed in volume relative to the total volume of the vaccine oil-in-water emulsion.

In some embodiments, the pharmaceutically acceptable carrier comprised in the vaccine oil-in-water emulsion according to the invention is the same as the aqueous solvent of the adjuvant oil-in-water emulsion. In preferred embodiments, the pharmaceutically acceptable carrier comprised in the vaccine oil-in-water emulsion according to the invention is water.

In some embodiments, the vaccine oil-in-water emulsion according to the invention is injectable, which means that its constituents and physical properties, such as its viscosity, are suitable for injection.

The vaccine oil-in-water emulsion according to the invention may comprise further components, such as those classically used in the formulation of vaccine oil-in-water emulsions. Preferably, said further components do not affect the safety nor the stability of the vaccine oil-in-water emulsions.

The vaccine oil-in-water emulsion according to the invention may comprise any further compound suitable for increasing and/or accelerating the immune response due to the antigen in the animal.

In an embodiment, the vaccine oil-in-water emulsion further comprises an immunostimulant. The immunostimulant may be selected from the group consisting of dimethyloctadecylammonium (DDA), saponin, Vitamin E, and an immunostimulant polymer such as a polyacrylic acid polymer. The amount of immunostimulant may be comprised between 0.5% and 5%, preferably about 1%, of the vaccine oil-in-water emulsion, in weight relative to the total volume of the vaccine oil-in-water emulsion.

### Use of the Vaccine Oil-In-Water Emulsion

A last object of the invention is a vaccine oil-in-water emulsion according to the invention for use as a medicament, preferably for use as a vaccine. The vaccine oil-in-water emulsion according to the invention is preferably for use in the treatment of porcine circovirus type 2 disease, of a Streptococcus suis infection such as porcine meningitis, porcine septicemia, porcine arthritis, porcine endocarditis, or porcine sudden death and/or of porcine enzootic pneumonia, more preferably porcine circovirus type 2 disease and/or porcine enzootic pneumonia.

Preferably, the vaccine oil-in-water emulsion according to the invention is used in a non-human mammal, more preferably in a porcine mammal, in particular in a pig or a piglet.

In some embodiments, the treatment of porcine circovirus type 2 disease, of a Streptococcus suis infection such as porcine meningitis, porcine septicemia, porcine arthritis, porcine endocarditis, or porcine sudden death and/or of porcine enzootic pneumonia is a prophylactic or preventive treatment.

The vaccine oil-in-water emulsion according to the invention may be administered to the animal by any suitable route. For instance, the vaccine oil-in-water emulsion according to the invention may be administered to the animal via percutaneous administration, via mucosal administration, via oral administration, or via injection, such as intradermal, intramuscular, subcutaneous, intravenous, or intraperitoneal injection. Preferably, the vaccine oil-in-water emulsion according to the invention is administered to the animal by injection.

One skilled in the art is able to adapt the dose of active agent, preferably of antigen, to be administered to the animal, and/or the frequency of administration, depending among others on the age, sex, weight, species and condition of the animal, the administration route and the nature of the active agent. The administration may be performed in one or several doses, preferably between one and three doses. In a preferred embodiment, the administration is performed in a single dose or in two doses.

Preferably, the vaccine oil-in-water emulsion according to the invention is sterile.

The present invention also relates to a method for the treatment of porcine circovirus type 2 disease, of a Streptococcus suis infection such as porcine meningitis, porcine septicemia, porcine arthritis, porcine endocarditis, or porcine sudden death and/or of porcine enzootic pneumonia, comprising the administration to a non-human mammal, preferably to a porcine mammal, of an efficient dose of a vaccine oil-in-water emulsion according to the invention.

The present invention also relates to the use of a vaccine oil-in-water emulsion according to the invention for the preparation of a medicament, preferably a vaccine, in particular a vaccine for the treatment of porcine circovirus type 2 disease, of a Streptococcus suis infection such as porcine meningitis, porcine septicemia, porcine arthritis, porcine endocarditis, or porcine sudden death and/or of porcine enzootic pneumonia.

The present invention also relates to a method for inducing an immune response in an animal, preferably a pig or a piglet, comprising administering to the animal vaccine oil-in-water emulsion according to the invention.

The present invention also relates to methods to immunize against, or to prevent or to reduce the symptoms caused by, infection of an animal with a pathogenic organism (for example, infection by a virus, bacteria, fungus, or protozoan parasite). Preferably, the pathogenic organism is porcine circovirus type 2, Streptococcus suis bacteria or Mycoplasma hyopneumoniae bacteria, more preferably porcine circovirus type 2 or Mycoplasma hyopneumoniae bacteria.

### Process for the Manufacture of the Vaccine Oil-In-Water Emulsion

The vaccine oil-in-water emulsion according to the invention may be prepared by any suitable process known in the art. In an embodiment, the vaccine oil-in-water emulsion is obtained by dilution of the adjuvant oil-in-water emulsion with a solution of the antigen in the pharmaceutically acceptable carrier, preferably in the aqueous pharmaceutically acceptable carrier.

The active agent, preferably the antigen, may be either incorporated during the formation of the emulsion, or incorporated later to the emulsion. In some embodiments, the active agent is incorporated to the emulsion just before use of the vaccine oil-in-water emulsion.

The size of the droplets in the emulsion may be from about 10 nm to about 5 μm. In an embodiment, the mean size of the droplets in the emulsion is about 60 nm.

### Kit

The present invention also relates to a kit comprising:
an active agent, preferably an antigen, as defined above, and
an adjuvant oil-in-water emulsion or a diluted adjuvant oil-in-water emulsion according to the invention.

The kit may further comprise a pharmaceutically acceptable carrier, preferably an aqueous pharmaceutically acceptable carrier, and/or instructions regarding the process for manufacturing the vaccine oil-in-water emulsion according to the invention.

As used herein, the term “comprising” and derivatives thereof must be understood as not limiting the presence of other components. In some embodiments, the term “comprising” and derivatives thereof may be understood as being more limitative, and meaning “essentially consisting of”, or even “consisting of”.

The invention will also be described in further detail in the following examples, which are not intended to limit the scope of this invention, as defined by the attached claims.

### Examples

### Example 1: Composition According to the Invention

The following oil adjuvant compositions were prepared by conventional methods. Composition A is an oil adjuvant composition according to the invention, and composition B is a comparative composition.

Composition A (w/w %)

| | |
|---|---|
| Marcol 52 (Exxon) | 82 |
| TEGO SMO V (Evonik) | 4 |
| BRIJ 010 (CRODA) | 14 |

Comparative composition B (w/w %)

| | |
|---|---|
| Marcol 52 (Exxon) | 82 |
| TEGO SMO V (Evonik) | 4.6 |
| Simulsol 2599 PHA VG (SEPPIC) | 13.4 |

Marcol™ 52 is a purified mixture of liquid saturated hydrocarbons.

TEGO® SMO V is a nonionic lipophilic surfactant comprising Sorbitan Oleate.

BRIJ 010 is a nonionic hydrophilic surfactant comprising Polyoxyethylene (10) oleyl ether.

Simulsol 2599 PHA VG is a hydrophilic nonionic surfactant comprising polyethylene glycol oleate (10).

### Example 2: Stability Study

A comparative stability study has been conducted on samples of Hyogen® vaccines formulated with different adjuvants, stored at 2-8° C. for 1 month. The antigen comprised in studied emulsions is a mycoplasma hyopneumoniae (M HYO) antigen.

Each tested formulation is obtained by replacing the adjuvant of the Hyogen® vaccine with oil-in-water emulsions of one of
Composition A,
Comparative composition B,
Comparative adjuvant composition LR3 oily phase disclosed in prior art WO2006/113373, and
Comparative adjuvant composition LR4 oily phase disclosed in prior art WO2006/113373.

The oily phase of LR3 comprises Brij®92 (Polyoxyethylene (2) oleyl ether), Brij®96 (Polyoxyethylene (10) oleyl ether), paraffin oil (Marcol® 82) and a preservative.

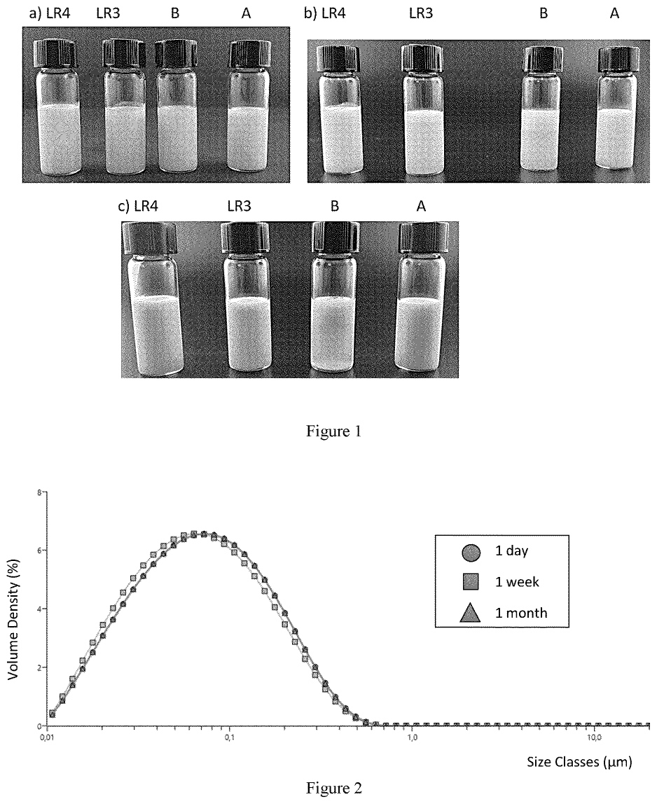

The oily phase of LR4 comprises Brij®92 (Polyoxyethylene (2) oleyl ether), Volpo® N5 (Polyoxyethylene (5) oleyl ether), paraffin oil (Marcol® 82) and a preservative. FIG. 1 presents the visual appearance of the studied compositions after 1 day (a), after 1 week (b) and after 1 month (c).
(a) After 1 day, the compositions look globally homogeneous, a slight creaming is only observed on the LR3 based vaccine composition.
(b) After 1 week, the same creaming is still visible on the LR3 based vaccine composition. The vaccine composition based on comparative adjuvant composition B has separated into 2 phases.
(c) After 1 month, the compositions based on LR3 and comparative composition B are almost completely separated into 2 phases. A slight creaming is now visible on the top of the composition based on LR4.

The size distribution of the particles in each composition has been measured at 1 day, 1 week, and 1 month by LASER diffraction with a Mastersizer 3000 particle size analyzer. The results are presented in table 1 below.

TABLE 1
Size distribution of each composition after 1 day, 1 week and 1 month

| | | | Comparative | | | | | |
|---|---|---|---|---|---|---|---|---|
| | Composition A | | composition B | | Composition LR3 | | Composition LR4 | |
| Sample | Dx(50) | Dx(90) | Dx(50) | Dx(90) | Dx(50) | Dx(90) | Dx(50) | Dx(90) |
| Time | (um) | (um) | (um) | (um) | (um) | (um) | (um) | (um) |
| 1 day | 0.070 | 0.210 | 0.059 | 0.136 | 1.370 | 5.630 | 0.129 | 0.530 |
| 1 week | 0.065 | 0.198 | 92.80 | 144.0 | 1.770 | 6.220 | 0.140 | 0.543 |
| 1 month | 0.070 | 0.209 | 107.0 | 185.0 | 1.710 | 7.390 | 0.123 | 0.478 |

FIGS. 2 to 5 presents the evolution of the size distribution of each composition from 1 day to 1 week.

FIG. 2: Composition A. The emulsion is perfectly stable in terms of particle size. The distribution is Gaussian, with an average value well below 1 μm, and does not evolve over time.

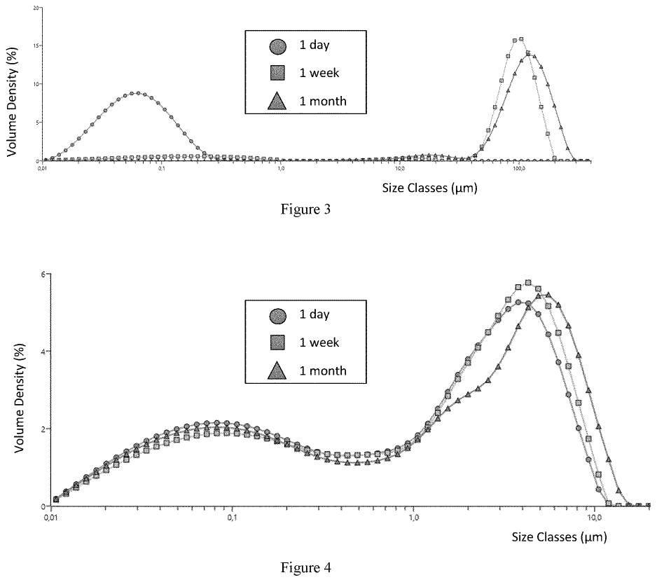

FIG. 3: Comparative composition B: The size of the particles increased from 60 nm to 100 μm after one week at 4° C. The size correlates well with the visual aspect. The emulsion was already broken after one week, and the breakage worsened after one month.

FIG. 4: Composition LR3: There are two populations of particles, the larger one being above 1 μm. The size correlates well with the visual aspect. The size distribution worsened over one month.

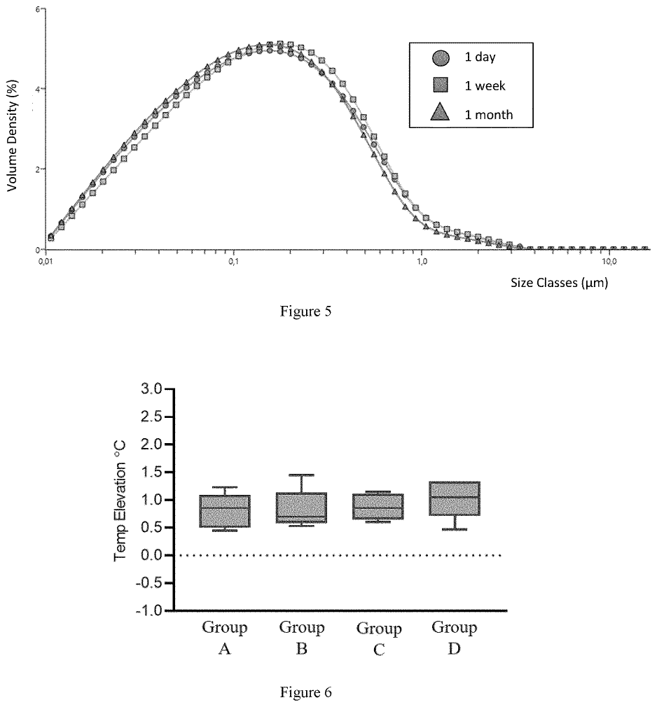

FIG. 5: Composition LR4: There are particles with a size exceeding 1 μm, which makes the size distribution of the particles uneven. This does not predict a good stability over time. The uneven size distribution remains over one month.

In summary, the vaccine composition based on Composition A according to the invention is the only oil-in-water emulsion whose particle size and visual appearance after one month's storage at 4 degrees are perfectly stable. This vaccine composition is expected to be stable for 1 to 2 years at 4 degrees.

### Example 3: Safety Study of a Hyogen Vaccine According to the Invention

The safety of a Hyogen® vaccine formulated with oil-in-water emulsion adjuvant composition A according to the invention was assessed in a field study performed in three farms in different provinces of China.

The reaction of pigs after being administered the formulations was assessed and scored according to the severity scoring scale detailed in table 2 below.

TABLE 2
Scoring scale of safety study

| Score of severity | 0 | 1 | 3 | 5 |
|---|---|---|---|---|
| Description | Good | Mild, | vomiting, | shock, |
| | no | depression/ | shaking, | moribund |
| | reaction | redness | walking | state, |
| | | only, | difficulty, | death |
| | | vomits | quick | |
| | | slightly | breathing | |
| | | and recovers | | |

Table 3 below presents the safety results for the formulation.

TABLE 3
Results of safety study

| Adjuvant | | Number of | Reaction | |
|---|---|---|---|---|
| type | Farm | animals | (number of animals) | Score |
| A | | 665 | 0 | |
| | Harbin | 100 | 0 | |
| | Hubei | 100 | 0 | |
| | Shandong | 465 | 0 | |

Among 665 pigs studied, none demonstrated a reaction after administration of the vaccine emulsion according to the invention.

This proves that the vaccine emulsions according to the invention are highly safe, in particular when used with a Mycoplasma hyopneumoniae antigen.

### Example 4 Safety Study of a PCV2 Vaccine According to the Invention

The safety of a PCV2 vaccine formulated with an oil-in-water emulsion adjuvant composition according to the invention (composition A of example 1) was assessed in a field study.

A total number of 20 PRRS negative PCV2 negative piglets aging at 2-3 weeks were used to investigate the overdose safety of 3 batches of PCV2 vaccine oil-in-water emulsions according to the invention, compared to a mock vaccine. Animals were randomly assigned to 4 groups A to D (5 in each group) before vaccination, among which 3 groups (groups A to C) injected with 4 mL of the vaccine oil-in-water emulsions according to the invention and 1 group (group D) injected with an equivalent dose of the mock vaccine. To investigate the safety performance, body temperature of each animal was collected daily from 3 days before to 6 days after vaccination. Additionally, temperature just before vaccination and 4 hours after vaccination was also collected. Clinical observation was conducted daily with attention to abnormal local and general reaction from the first to 14th days after vaccination.

Results showed that a transient body temperature increase was observed within the first day post vaccination and all animals recovered afterwards. Maximum temperature increase of each group did not exceed 1.5° C., with average between 0.8 and 0.9° C. FIG. 6 and Table 4 below present the temperature data for each group. No significant difference was found among the groups. Beside the transient temperature increase, no general nor local adverse reactions were observed during the study. The safety of the 3 batches of vaccine oil-in-water emulsions according to the invention is confirmed.

Maximum temperature increase of each animal was calculated for ANOVA test. No significant difference was found among groups (p=0.701).

TABLE 4
Summarized data of temperature increase
monitoring in each group

| Group | N | Mean | SD | Minimum | Maximum |
|---|---|---|---|---|---|
| A | 5 | 0.806 | 0.3141 | 0.5 | 1.2 |
| B | 5 | 0.826 | 0.3645 | 0.5 | 1.5 |
| C | 5 | 0.876 | 0.2369 | 0.6 | 1.2 |
| D | 5 | 1.026 | 0.3537 | 0.5 | 1.3 |

### Example 5: Efficacy of Adjuvant Compositions According to the Invention

A comparative efficacy study has been conducted on piglets, PCV2 vaccine formulations comprising different adjuvants were tested.

80 piglets, pure breed (largewhite, landrace, Genesus) were divided in 4 groups of 20 animals.
Group A was injected with the reference product Circoflex (Porcine circovirus type 2 ORF2 protein with Carbomer as adjuvant) and challenged 21 days post vaccination with a PCV2 strain SEVC1702;
Group B was injected with Placebo (Oil-in-water emulsion adjuvant of the invention obtained from the oil composition Composition A of example 1+phosphate-buffered saline PBS) and challenged 21 days post vaccination with a PCV2 strain SEVC1702;
Group C was injected with a PCV2 vaccine comprising Porcine circovirus type 2 ORF2 protein and an oil-in-water emulsion adjuvant according to the invention, and challenged 21 days post vaccination with a PCV2 strain SEVC1702
Group D was injected with a PCV2 vaccine comprising Porcine circovirus type 2 ORF2 protein and an oil-in-water emulsion adjuvant according to the invention, and challenged 14 days post vaccination with a PCV2 strain SEVC1702.

Piglets of each group were necropsied 28 days after the challenge. (at day 49 for groups A, B and C and at day 42 for group D).

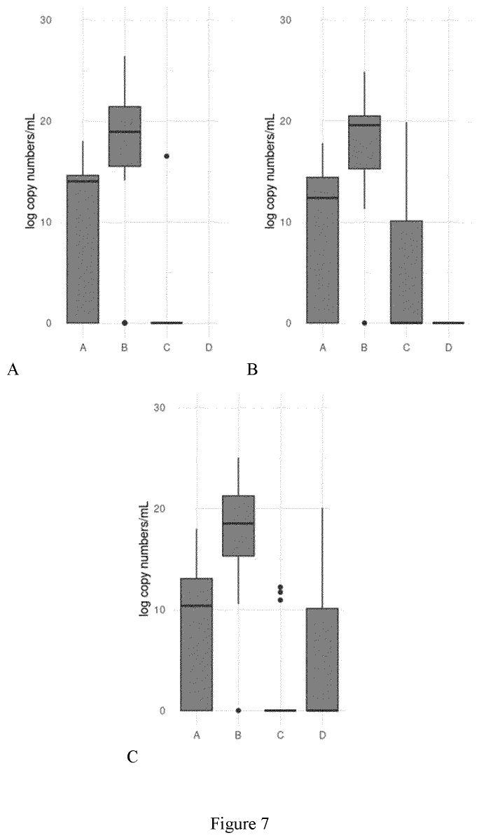

FIGS. 7A, B and C respectively show the viremia of each group at day 14, 21 and 28 after the challenge. Each group reduced viremia significantly in comparison with the placebo (group B).

Adjuvant according to the invention (group C and D), has significantly lower viremia than the reference product Circoflex.

Nasal swabs were collected for qPCR on days 7, 14, 21, and 28 after tapping, respectively. The test results were Log transformed and analyzed.

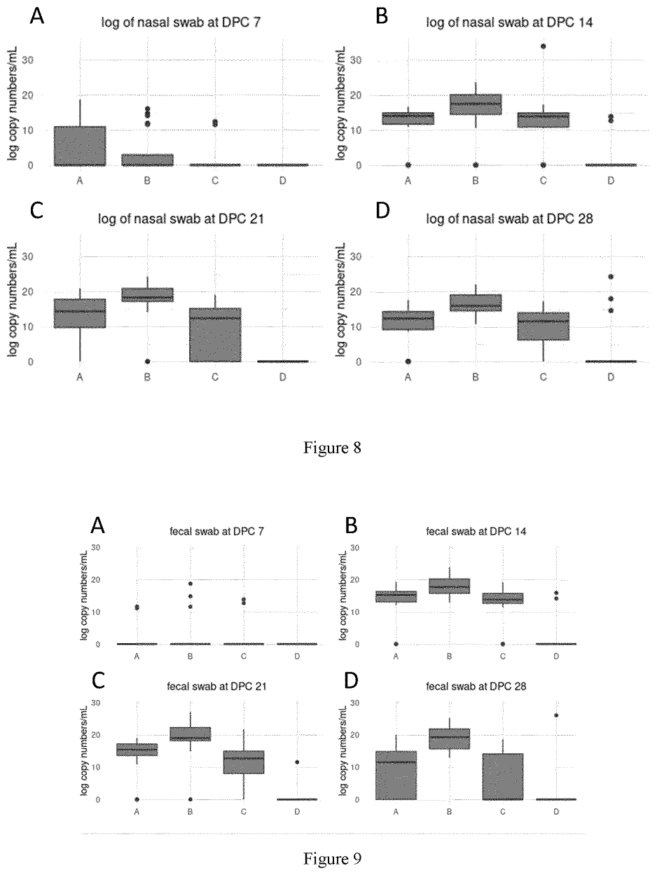

FIGS. 8A, B and C respectively show the log of nasal swab at respectively day 7, 14, 21 and 28. Group D excreted significantly less virus via the nasal passages than group A.

Anal swabs were collected on days 7, 14, 21 and 28 after tapping for qPCR assay, respectively. The test results were Log transformed and analyzed.

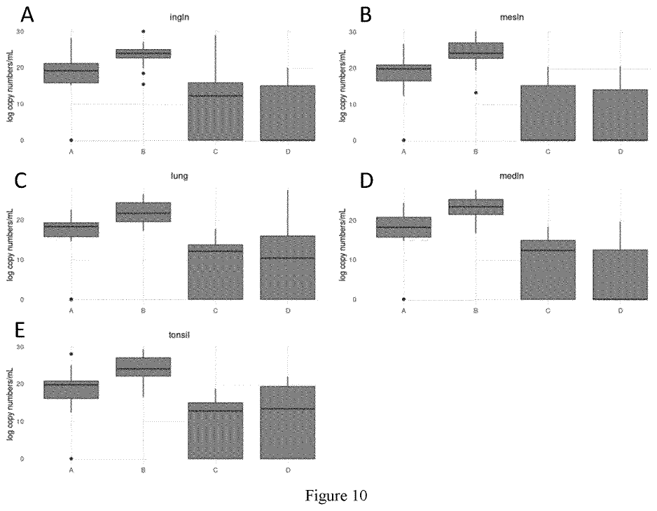

FIGS. 9A, B, C, and D respectively show the log of nasal swab at day 7, 14, 21 and 28. The virus excreted via feces was significantly lower in group D than in group A on days 14, 21, and 28 after attack.

After euthanasia of all animals on day 28 after attack (DPC28), tonsils, mediastinal lymph nodes, mesenteric lymph nodes, inguinal lymph nodes and lungs were collected by dissection. Small pieces of each tissue about the size of a grain of rice were cut, and total DNA extraction of the tissues were performed directly with a genome extraction kit. Afterwards, quantification of viral nucleic acids was performed with a qPCR kit. The test data were Log-transformed and analyzed for between-group differences using the Kruskal-Wallis test, followed by two-by-two comparisons of between-group differences using the Wilcoxon rank-sum test.

FIGS. 10A, B, C, D and E show log quantification of the virus at day 28 for respectively inguinal lymph nodes, mesenteric lymph nodes, lung, mediastinal lymph nodes and tonsil lymph nodes.

Both Group A and Group C were able to significantly inhibit the viral load in lymph node tissues, lungs, and tonsils compared to the placebo group (Group B). Between the groups, Groups C and D suppressed the viral load in the lymph nodes and tonsils better than Group A, and the difference was significant.

Finally, PCV2-specific antibody levels were evaluated using a commercial ELISA kit.

The results showed that all vaccine-immunized groups could induce the production of specific antibodies in the animal organism, except for the placebo group. Animals in the placebo group did not show any antibody turnover prior to the tapping. Compared with the placebo group, higher antibody levels could be stimulated in a shorter period of time in the immunized group animals after the tapping.

### Conclusion:

The results showed that both PCV2 vaccines comprising the adjuvant of the invention were able to significantly reduce the viral load in the blood, the amount of virus excreted via the nose and anus, as well as significantly reduce PCV2 virus levels in the lymph nodes, tonsils and lungs, compared to placebo. The two batches of vaccine comprising the adjuvant of the invention used in this study significantly increased the level of inhibition of nasal and fecal elimination and the level of inhibition of viral load in lymph nodes and tonsils compared to reference products indicating that the vaccine comprising the adjuvant of the invention provides superior protection against the 2d genotype PCV2 strain.

The clinical safety of the two laboratory vaccine batches was also observed in this study. After immunization, neither batch of vaccine produced severe acute reactions, nor were any local adverse reactions at the injection site observed.

### Example 6: Vaccinal Compositions Comprising IdeSsuis Antigen and an Adjuvant Composition According to the Invention

Streptococcus suis (S. suis) is a major porcine pathogen causing meningitis, arthritis and several other pathologies. The immunoglobulin M-degrading enzyme of Streptococcus suis, IdeSsuis, is a highly protective antigen against Streptococcus suis infection.

One hundred 7 to 10-week-old piglets were grouped into 5 groups.

The piglets of groups 1-5 were vaccinated twice (on DO and D21) with a recombinant IdeSsuis IgM protease protein as mentioned in the Test items listed in Table 5 below. Control group 6 was treated with physiological NaCl solution. The O/W adjuvant of the invention corresponds to the O/W emulsion obtained from the oil composition Composition A of example 1 and phosphate-buffered saline PBS.

| | |
|---|---|
| Group 1: | 20 rU/mL rldeSsuis + 50% o/w adjuvant |
| | (Montanide ISA201) |
| Group 2: | 20 rU/mL rldeSsuis + 30% o/w adjuvant |
| | (Montanide ISA 201) |
| Group 3: | 20 rU/mL rIdeSsuis + 50% o/w adjuvant |
| | of the invention |
| Group 4: | 20 rU/mL rIdeSsuis + 30% o/w adjuvant |
| | of the invention |
| Group 5: | 20 rU/mL rIdeSsuis + 30% o/w adjuvant |
| | with dimethyldioctadecylammonium |
| | bromide DDA (Emulsigen ® D) |
| Control (Group 6) | NaCl physiological |

The local and systemic reactions were evaluated at D7, D10, D14, D24, D27, D31 and D35. Clinical health was examined daily.

On study days-1, 21 and 35, blood samples were taken from all animals. The study ended at study day 35.

### Results

### 1. Local Tolerance

Local tolerance was observed (by palpation) at D7, D10, D14, D24, D27, D31 and D35.

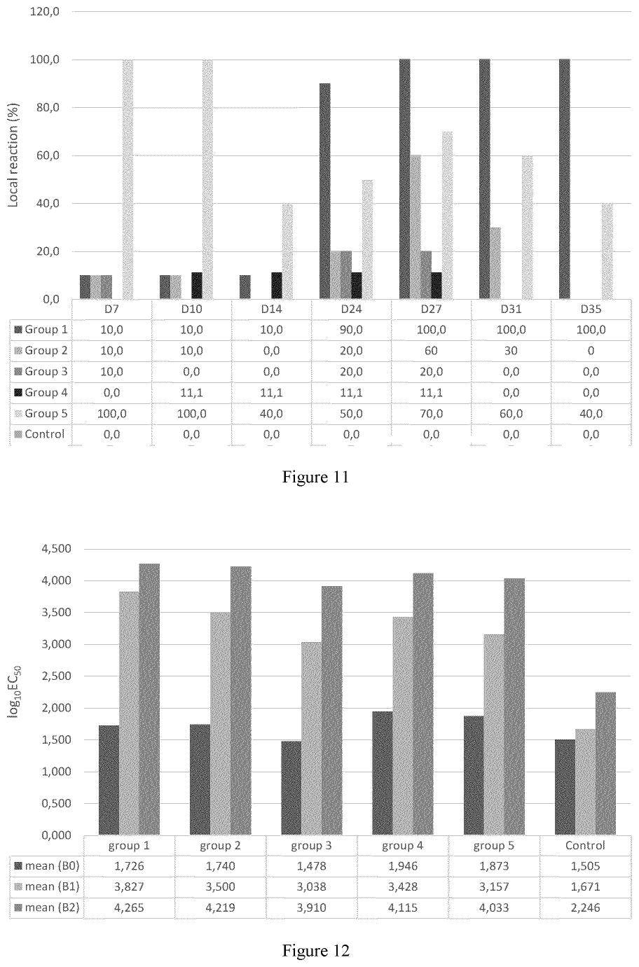

Percentage of local reaction per day are represented in FIG. 11.

Groups 3 and 4, which were vaccinated with an adjuvant emulsion according to the invention showed better or equal tolerance than the other groups at any day.

Between D24 and D31, Groups 3 and 4 showed better local reaction results than any other groups.

Groups 1 and 2 showed an important difference in local reaction depending on the adjuvant concentration. Such difference is not visible for the adjuvant of the invention.

In conclusion, adjuvant of the invention shows less local reactions than reference adjuvants and shows a better safety due to the specific used formulation.

### 2. Antibody Titers

Antibody titers against the antigen in the vaccine preparations were determined from serum samples using ELISA. Results are presented in FIG. 12. B0 corresponds to before the first vaccination, B1 between the first and the second vaccinations and B2 2 weeks after the second vaccination.

### Results

The use of an adjuvant of the invention to produce a vaccine against Streptococcus suis showed similar antibody development than the use of reference adjuvants.

1. An oil adjuvant composition comprising:
at least one oil,
at least one nonionic lipophilic surfactant, which is a sorbitan ester or a mannitan ester, and
at least one nonionic hydrophilic surfactant, which is an ethoxylated fatty alcohol.

2. The oil adjuvant composition according to claim 1, wherein the oil comprises at least one mineral oil or a combination of at least two mineral oils.

3. The oil adjuvant composition according to claim 2, wherein the at least one mineral oil is selected from the group consisting of paraffin oil, pristane, and any mixture thereof.

4. The oil adjuvant composition according to claim 1, wherein each sorbitan ester or mannitan ester is a sorbitan ester.

5. The oil adjuvant composition according to claim 1, wherein each sorbitan ester or mannitan ester is a mannitan ester.

6. The oil adjuvant composition according to claim 1, wherein the ethoxylated fatty alcohol is selected from the group consisting of polyoxyethylene (23) lauryl ether, polyoxyethylene (20) cetyl ether, polyoxyethylene (10) oleyl ether, polyoxyethylene (20) oleyl ether, polyoxyethylene (21) stearyl ether, polyoxyethylene lauryl ether, eicosaethylene glycol octadecyl ether, triethylene glycol oleyl ether, and any mixture thereof.

7. The oil adjuvant composition according to claim 1, wherein the oil adjuvant composition comprises:
from 76% to 92% of oil,
from 1% to 7% of at least one nonionic lipophilic surfactant, which is a sorbitan ester or a mannitan ester, and
from 7% to 17% of at least one nonionic hydrophilic surfactant, which is an ethoxylated fatty alcohol,
wherein the percentages are expressed in weight relative to the total volume of the oil adjuvant composition.

8. The oil adjuvant composition according to claim 7, wherein the oil adjuvant composition comprises:
about 82% of oil,
about 4% of at least one nonionic lipophilic surfactant, which is a sorbitan ester or a mannitan ester, and
about 14% of at least one nonionic hydrophilic surfactant, which is an ethoxylated fatty alcohol,
wherein the percentages are expressed in weight relative to the total volume of the oil adjuvant composition.

9. An adjuvant oil-in-water emulsion, comprising 40% to 70% of an oil adjuvant composition according to claim 1, and 30% to 60% of an aqueous solvent,
the oil adjuvant composition being dispersed in the aqueous solvent,
wherein the percentages are expressed in volume relative to the total volume of the adjuvant oil-in-water emulsion.

10. A vaccine oil-in-water emulsion, comprising an adjuvant oil-in-water emulsion according to claim 9, at least one antigen, and a pharmaceutically acceptable carrier.

11. The vaccine oil-in-water emulsion according to claim 10, wherein the vaccine oil-in-water emulsion comprises from 10 to 80% of the adjuvant oil-in-water emulsion, and from 20% to 90% of pharmaceutically acceptable carrier,
wherein the percentages are expressed in volume relative to the total volume of the vaccine oil-in-water emulsion.

12. The vaccine oil-in-water emulsion according to claim 10, wherein the at least one antigen is selected from the group consisting of a porcine circovirus type 2 (PCV2) antigen, a Streptococcus Suis antigen, a Mycoplasma hyopneumoniae antigen and a combination thereof.

13. The vaccine oil-in-water emulsion according to claim 10, for use as a medicament, preferably for use as a vaccine.

14. The vaccine oil-in-water emulsion according to claim 10 for use in the treatment of porcine circovirus type 2 disease, a Streptococcus suis infection and/or porcine enzootic pneumonia.

15. The vaccine oil-in-water emulsion for use according to claim 13, wherein the vaccine oil-in-water emulsion is administered, to an animal being a pig or a piglet.
